(19) United States
(12) Patent Application Publication
(10) Pub. No.: US 2011/0207807 A1
(43) Pub. Date: Aug. 25, 2011

(54) NEW USE FOR HOMOISOFLAVONE OR A SALT THEREOF

(75) Inventors: Tae Yoon Kim, Seoul (KR); Dong Heon Shin, Daejeon (KR)

(73) Assignee: Catholic University Industry Academy Cooperation Foundation, Seoul (KR)

(21) Appl. No.: 13/061,836

(22) PCT Filed: Sep. 2, 2009
(86) PCT No.: PCT/KR2009/004937

§ 371 Date: Apr. 28, 2011

(30) Foreign Application Priority Data
Sep. 2, 2008 (KR) 10-2008-0086418
Sep. 2, 2008 (KR) 10-2008-0086424

(51) Int. Cl. A61K 31/352; C07D 311/22; A61P 29/00; A61P 37/08; A61P 17/00; A61P 17/06; A61P 17/10

(52) U.S. Cl. 514/456; 549/401

(57) ABSTRACT

The present disclosure relates to a new use of homoisoflavanone or a salt thereof. More particularly, it relates to a pharmaceutical composition for the prevention and treatment of inflammatory diseases or allergic diseases comprising homoisoflavanone represented by Chemical Formula 1 or a salt thereof, a use of homoisoflavanone or a salt thereof in the manufacture of an agent for the preventing and treating of inflammatory diseases or allergic diseases, and a method for treating inflammatory diseases or allergic diseases including administering an effective dose of homoisoflavanone or a salt thereof to a subject in need thereof.

### TECHNICAL FIELD

This application claims priority to Korean Patent Application No. 10-2008-0086418 filed on Sep. 2, 2008, which is hereby incorporated by reference herein.

The present disclosure relates to a new use of homoisoflavanone or a salt thereof. More particularly, it relates to a pharmaceutical composition for the prevention and treatment of inflammatory diseases or allergic diseases containing homoisoflavanone represented by Chemical Formula 1 or a salt thereof, a use of homoisoflavanone or a salt thereof in the manufacture of an agent for the prevention and treatment of inflammatory diseases or allergic diseases, and a method for the treatment of inflammatory diseases or allergic diseases comprising administering an effective amount of homoisoflavanone or a salt thereof to a subject in need thereof.

### BACKGROUND ART

Homoisoflavanone isolated from Cremastra appendiculata Makino is known to be involved in anti-angiogenesis in chick embryo and inhibit cell cycle arrest by suppressing the expression of cdc2. However, there is no substantial study result about the medicinal effect of this substance.

Inflammatory response refers to the complex physiological response of vascular tissues to harmful stimuli, such as damages, bacteria, fungi, viruses, etc., including enzymatic activation caused by various inflammatory mediators and immunocytes, secretion of inflammatory mediators, infiltration of body fluid, cell migration, tissue destruction, or the like. As a result, such symptoms as erythema, edema, fever and pain are accompanied. Inflammatory response is a protective attempt by the organism to remove exogenous source of infection, regenerate damaged tissues and restore biological functions. However, excessive or prolonged inflammatory response, which may be caused by unremoved antigens or internal substances, can lead to damaged mucosa, tissue destruction, or such diseases as cancer, inflammatory skin disease, inflammatory bowel disease, arthritis, etc.

Until now, antihistamines, vitamin ointments, NSAID immune inhibitor and adrenocorticotropic hormones have been commonly used to treat inflammatory diseases. However, these drugs provide temporary effect only and sometimes are associated with severe side reactions. Accordingly, there is a need for the development of new substances effective in treating inflammatory diseases without side effects. For example, ML-3000 (licofelone) which is under development by Merck is a dual inhibitor suppressing the expression of COX-1/COX-2 and 5-LOX, and is known to have anti-inflammatory and analgesic effects, being effective in hypersensitivity, allodynia, fever, myocardial infarction and osteoarthritis, with less gastrointestinal side effects (Kulkarni S et al., 2007; 7(3) 251-63, Current Topics in Medicinal Chemistry). Quercetin, which is found in many plants, has anti-histamine, anti-cardiovascular disease, anti-cancer, anti-inflammatory, anti-carcinogenic (against prostate cancer, ovarian cancer, breast cancer, gastric cancer, etc.) and heavy metal detoxifying effects.

Allergic diseases refer to a hypersensitive reaction of the immune system to normally harmless external substances (allergens). The allergic diseases are immunological disorders frequently occurring due to environmental factors such as temperature, humidity, climate, environmental pollution, working environment, or the like, but a fundamental cure is not developed as yet.

Most of the currently developed antiallergenic agents inhibit the various signaling pathways of the chemical transmitters produced by inflammatory cells. When mast cells can be stimulated to degranulate due to allergens, the chemical substances stored in the granules of the mast cells, such as histamine, heparin and proteases are released, causing immediate allergic and inflammatory reactions. Most of currently available antiallergenic agents are anti-histamine drugs inhibiting such chemical substances and, in severe cases, steroid agents are used together. However, those synthetic drugs do not provide complete treatment, resulting in decreased effect and severe systemic adverse effects upon prolonged use.

The skin is the outermost organ directly exposed to the external environment. When exposed to excessive UV radiation, pollutants, or the like, skin irritation and inflammatory reactions such as erythema, edema, pimple, itching, etc. are induced. Such skin troubles are not only aesthetically unattractive, but also the substances produced during the inflammatory reactions are known to induce skin pigmentation and accelerate the damage of skin elastic fibers, resulting in skin wrinkling.

Thus, various substances are developed to suppress skin inflammations. However, they are mostly antiinflammation agents such as anti-histamine agents, vitamin ointments or adrenocortical hormone agents, whose effect is only transient. Often, they result in severe side reactions after long-term use, making them.

Further, since the skin is exposed to various external stimuli, allergic reactions may occur easily. Allergy refers to a hypersensitive reaction of the immune system to normally harmless external substances (allergens). The allergic diseases are immunological disorders frequently occurring due to environmental factors such as temperature, humidity, climate, environmental pollution, working environment, or the like, but a fundamental cure is not developed as yet.

As in the inflammatory reactions, the occurrence of allergic reactions on the skin is not only aesthetically unattractive, but also it may result in skin pigmentation and skin wrinkling. Although anti-histamine agents or steroid agents, which inhibit histamine, heparin and proteases, are administered together to prevent and treat the allergic reactions, their effect decreases after long-term use. Further, they are inapplicable to cosmetics because of severe systemic adverse effects.

### Disclosure

### Technical Problem

The inventors of the present disclosure have carried out researches on the physiological functions of homoisoflavanone. As a result, they have found out that it has an unknown function of inhibiting inflammatory reactions or allergic reactions. Thus, the present disclosure is directed to providing a pharmaceutical composition for the prevention and treatment of inflammatory or allergic diseases comprising homoisoflavanone, an use thereof and treating method, and a cosmetic composition for preventing and improving inflammatory or allergic diseases comprising a homoisoflavanone.

Accordingly, the object of the present invention is to provide a novel use of homoisoflavanone or a salt thereof.

### Technical Solution

To achieve the above object, the present invention provides a pharmaceutical composition for preventing and treating inflammatory diseases comprising homoisoflavanone or a pharmaceutically acceptable salt thereof as an effective ingredient.

To achieve another object, the present invention provides a pharmaceutical composition for preventing and treating allergic diseases comprising homoisoflavanone or a pharmaceutically acceptable salt thereof as an effective ingredient.

To achieve still another object, the present invention provides a cosmetic composition for preventing and improving inflammation comprising homoisoflavanone or a pharmaceutically acceptable salt thereof as an effective ingredient.

To achieve still another object, the present invention provides a cosmetic composition for preventing and improving allergy comprising homoisoflavanone or a salt thereof as an effective ingredient.

To achieve still another object, the present invention provides use of homoisoflavanone represented by Chemical Formula 1 or a pharmaceutically acceptable salt thereof for the preparation of an agent for preventing and treating inflammatory diseases.

To achieve still another object, the present invention provides a method for treating inflammatory diseases comprising administering to a subject in need thereof an effective amount of homoisoflavanone represented by Chemical Formula 1 or a pharmaceutically acceptable salt thereof.

To achieve still another object, the present invention provides use of homoisoflavanone represented by Chemical Formula 1 or a pharmaceutically acceptable salt thereof for the preparation of an agent for preventing and treating allergic diseases.

To achieve still another object, the present invention provides a method for treating allergic diseases comprising administering to a subject in need thereof an effective amount of homoisoflavanone represented by Chemical Formula 1 or a pharmaceutically acceptable salt thereof.

To achieve still another object, the present invention provides use of homoisoflavanone represented by Chemical Formula 1 or a salt thereof for the preparation of cosmetics for preventing and improving skin inflammation.

To achieve still another object, the present invention provides a method for improving skin inflammation comprising applying to a subject in need thereof an effective amount of homoisoflavanone represented by Chemical Formula 1 or a salt thereof.

To achieve still another object, the present invention provides use of homoisoflavanone represented by Chemical Formula 1 or a salt thereof for the preparation of cosmetics for preventing and improving allergy.

To achieve still another object, the present invention provides a method for improving allergy comprising applying to a subject in need thereof an effective amount of homoisoflavanone represented by Chemical Formula 1 or a salt thereof.

Hereafter, the present invention will be described in more detail.

In the pharmaceutical composition, cosmetic composition, use and method of the present invention, it is characterized that they comprise homoisoflavanone or a pharmaceutically acceptable salt thereof or use them for preparation of an agent for treatment or are administered to a subject or are applied to a subject.

The homoisoflavanone of the present disclosure is represented by Chemical Formula 1. It may be isolated and purified from natural products, purchased commercially, or chemically synthesized according to a method known in the related art:

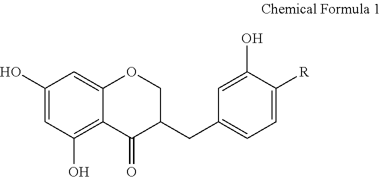

(wherein R is H, OH or OCₙH₂ₙ₊₁ (n is from 1 to 6)).

Specifically, the homoisoflavanone may be isolated/purified from Cremastra appendiculata. The homoisoflavanone according to the present disclosure may be extracted by a method known in the art, such as solvent extraction, chromatographic extraction, or the like.

In an example of the present disclosure, inhibitory effect of homoisoflavanone on intracellular reactive oxygen species induced by UV radiation was investigated. As a result, the reactive oxygen species generated by UV radiation were decreased by homoisoflavanone treatment, which suggests that homoisoflavanone has an antioxidative effect (see Example 1).

In another examples of the present disclosure, inhibitory effect of homoisoflavanone on the COX-2 expression induced by UV and on the expression of pro-inflammatory cytokines interleukin-6 (IL-6), IL-8 and tumor necrosis factor-α (TNF-α) induced by UV was investigated. As a result, homoisoflavanone was effective in reducing inflammatory reactions caused by the increased expression of COX-2 and the pro-inflammatory cytokines IL-6, IL-8 and TNF-α induced by UV radiation (see Examples 2 and 3).

In another example of the present disclosure, inflammatory reactions and skin damage induced by UV, COX-2 expression induced by UV, and expression of the pro-inflammatory cytokines IL-6 and TNF-α induced by UV were investigated in an animal model. As a result, homoisoflavanone relieved UV-induced inflammatory reactions and had an effect of reducing the increased expression of COX-2 and the pro-inflammatory cytokines IL-6 and TNF-α. This suggests that homoisoflavanone has an effect of inhibiting inflammatory reaction induced by UV radiation in vivo (see Examples 4 and 5).

In another example of the present disclosure, inhibitory effect of homoisoflavanone on the expression of the pro-inflammatory factor leukotriene, the cytokines IL-6 and IL-8, and COX-2 induced by phorbol 12-myristate 13-acetate and A23187 was investigated in mast cells. As a result, homoisoflavanone inhibited expression of COX-2, 5-LOX which translocated to the nuclear membrane, phosphorylated cPLA₂, leukotrienes B4 and C4, and the pro-inflammatory cytokines IL-6 and IL-8, expression of which was induced by phorbol 12-myristate 13-acetate and A23187. This suggests that homoisoflavanone has an effect of suppressing inflammatory reactions (see Examples 6 and 7).

In another example of the present disclosure, inhibitory effect of homoisoflavanone on the degranulation of mast cells induced by phorbol 12-myristate 13-acetate and A23187 was investigated. As a result, homoisoflavanone inhibited the degranulation of the mast cells induced by phorbol 12-myristate 13-acetate and A23187 and also inhibited the degranulation induced by antigens (IgE). This suggests that homoisoflavanone has an effect of suppressing inflammatory reactions (see Example 8).

In another example of the present disclosure, inhibitory effect of homoisoflavanone on the proliferation of T cells was investigated by mixed lymphocyte reaction. As a result, addition of homoisoflavanone at a concentration of 0.25 μg/mL or higher resulted in effective inhibition of T cell growth in a concentration-dependent manner (see Example 9).

In another example of the present disclosure, inhibitory effect of homoisoflavanone on the skin inflammatory reaction induced with UV radiation or croton oil was investigated. As a result, homoisoflavanone had a concentration-dependent effect of inhibiting ear edema and skin inflammatory reactions of 50% or more (see Example 10).

Accordingly, the present disclosure provides a pharmaceutical composition for the prevention and treatment of inflammatory diseases containing homoisoflavanone or a pharmaceutically acceptable salt thereof. In addition, there is provided a use of homoisoflavanone or a pharmaceutically acceptable salt thereof in the manufacture of a drug for the prevention and treatment of inflammatory diseases. Further, there is provided a method for the treatment of inflammatory diseases including administering an effective dose of homoisoflavanone or a pharmaceutically acceptable salt thereof to a subject in need thereof.

The homoisoflavanone according to the present disclosure may be used as it is or in the form of a pharmaceutically acceptable salt. As used herein, the phrase ‘pharmaceutically acceptable’ means that the salt is physiologically acceptable and, when administered to human, usually does not cause allergic reactions or similar adverse reactions. Specifically, it may be an acid addition salt formed from a pharmaceutically acceptable free acid. The free acid may be an organic or inorganic acid. Non-limiting examples of the organic acid include citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, glutamic acid and aspartic acid. And, non-limiting examples of the inorganic acid include hydrochloric acid, bromic acid, sulfuric acid and phosphoric acid.

The inflammatory diseases to which the compound of the present invention can be applied may include inflammatory skin disease, inflammatory bowel disease such as Crohn's disease and ulcerative colitis, peritonitis, osteomyelitis, phlegmon, meningitis, encephalitis, pancreatitis, trauma-induced shock, bronchial asthma, allergic rhinitis, cystic fibrosis, stroke, acute bronchitis, chronic bronchitis, acute bronchiolitis, chronic bronchiolitis, osteoarthritis, gout, spondyloarthropathy, ankylosing spondylitis, Reiter's syndrome, psoriatic arthropathy, enteropathic spondylitis, juvenile arthropathy, juvenile ankylosing spondylitis, reactive arthropathy, inflammatory arthritis, post-inflammatory arthritis, gonococcal arthritis, tuberculous arthritis, viral arthritis, mycotic arthritis, syphilitic arthritis, Lyme disease, vasculitic syndrome-related arthritis, polyarteritis nodosa, hypersensitivity vasculitis, Luegenec's granulomatosis, rheumatoid polymyalgia, articular cell arteritis, calcium crystal deposition arthropathy, pseudogout, nonarticular rheumatism, bursitis, tenosynovitis, epicondylitis (tennis elbow), neuropathic joint disease (Charco and joint), hemarthrosis, Henoch-Schönlein purpura, hypertrophic osteoarthropathy, multicentric reticulohistiocytoma, surcoilosis, hemochromatosis, sickle-cell anemia and other hemoglobinopathies, hyperlipoproteinemia, hypogammaglobulinemia, familial Mediterranean fever, Behçet disease, systemic lupus erythematosus, recurrent fever, psoriasis, multiple sclerosis, septicemia, septic shock, multiple organ dysfunction syndrome, acute respiratory distress syndrome, chronic obstructive pulmonary disease, rheumatoid arthritis, acute lung injury, bronchopulmonary dysplasia, and the like, but not limited thereto.

Further, the inflammatory skin disease may include skin inflammation, acute/chronic eczema, contact dermatitis, atopic dermatitis, seborrheic dermatitis, lichen simplex chronicus, intertrigo, exfoliative dermatitis, papular urticaria, lichen planus, acute/chronic lichenoid dermatitis, irritant dermatitis, parapsoriasis, pityriasis rosea, panniculitis, solar dermatitis, exfoliative dermatitis, mastocytosis, psoriasis, acne, and the like, but not limited thereto.

A pharmaceutical composition of the present invention may comprise homoisoflavanone or a pharmaceutically acceptable salt thereof alone or together with pharmaceutically acceptable carrier, excipient or dilutent. The composition of the present invention may comprise homoisoflavanone or a pharmaceutically acceptable salt thereof at the range of 0.0001% to 99.999%.

A pharmaceutically acceptable carrier, for example, carriers for the parenteral or oral preparations may be included. The carriers for the oral preparations may comprise lactose, starch, cellulose derivatives, magnesium stearate, stearic acid. In addition, the carriers for the parenteral preparations may comprise water, oil, saline, aqueous glucose and glycol, and stabilizers and preservatives. The examples of the stabilizers may be antioxidant such as sodium hydrogen sulfite, sodium sulfite, and ascorbic acid. The examples of the preservatives may be benzalkonium chloride, methyl- or prophyl-paraben, and chlorobutanol. Another pharmaceutically acceptable carriers are disclosed in Remington's Pharmaceutical Sciences, 19th ed., Mack Publishing Company, Easton, Pa., 1995.

A pharmaceutical composition for preventing and treating skin disease of the present invention may be administered to mammals including human beings by any routes. For example, it may be administered parenterally or orally. For parenteral administration, but not limited thereto, it may be administered parenterally, by intravenous, intramuscular, intraarterial, intramarrow, subdural, intracardiac, intracutaneous, subcutaneous, intraperitoneal, intranasal, gastrointestinal tracts, parenteral, sublingual or rectum. Preferably, a pharmaceutical composition of the present invention may be administered by intracutaneous. The said ‘intracutaneous’ refers to transfer effective amount of an active ingredient which is comprised in the composition for preventing and treating inflammatory disease by administered a pharmaceutical composition of the present invention into the cell or the skin. For example, an injectable form of the pharmaceutical composition of the present invention may be administered by lightly pricking the skin with 30-gauge injection needle, or applying directly into the skin.

A pharmaceutical composition of the present invention may be prepared in the form of oral preparation or parenteral preparation according to the described above.

In case of the formulation for oral administration, the composition of the present invention may be formulated into powders, granules, tablets, pills, and sugar-coated tablets, capsules, liquids, gels, syrups, slurrys, and emulsions by using the method known in the art. For example, preparations for oral administration may be harvested in the form of tablets or sugar-coated tablets by mixing an effective component with a solid excipient, grinding, and adding appropriate supplemental agents, then manufacturing a form of granular mixture. For examples of appropriate excipient, it may comprise sugars comprising lactose, dextrose, sucrose, sorbitol, mannitol, xylitol, erythritol and maltitol, starches comprising corn starch, wheat starch, rice starch and potato starch, celluloses comprising cellulose, methyl cellulose, sodium carboxymethylcellulose and hydroxypropylmethylcellulose, and fillers comprising gelatin and polyvinylpyrrolidone. And, if desired, it may comprise cross-linked polyvinylpyrrolidone, agar, alginic acid or sodium alginate as an solutionizer. Further, the inventive pharmaceutical composition may comprise anti-coaglutinating agent, lubricant, wetting agents, flavors, emulsifying agents and antiseptics additionally.

In case of pharmaceutical formulations for parenteral administration, it may be prepared in the forms of injectable preparations, creams, lotions, ointments, oils, humectant, gels, aerosol, and nasal inhalations by the method well known in the art. The formulation of the above-mentioned is well described in Remington's Pharmaceutical Science, 15th Edition, 1975, Mack Publishing Company, Easton, Pa. 18042, Chapter 87: Blaug, Seymour.

Total effective amount of homoisoflavanone of the present invention may be administered to a patient with a single dose, or may be administered with multiple doses by fractionated treatment protocol. The pharmaceutical compositions of the present invention may contain variable amount of effective ingredient according to the disease severity. In case of parenteral administration, the effective amount of homoisoflavanone of the present invention is preferably about 0.001 ug to 1,000 mg/kg body weight/day, most preferably 0.01 ug to 100 mg/kg body weight/day. However, the dose of the said homoisoflavanone may be suitably determined by considering various factors, such as age, body weight, health condition, sex, disease severity, diet and excretion of a subject in need of treatment, as well as administration time and administration route. Therefore, when those are considered, skilled person in the art may determine appropriate dose of the said homoisoflavanone for a certain use for preparing reagent for preventing and treating inflammatory disease. A pharmaceutical composition of the present invention may not limit formulations, administration routes, and administration methods as long as they show the effect of the present invention.

As used herein, the term “effective amount” refers to an amount showing a treating effect against the relevant diseases in a subject and the “subject” refers to a mammal and, preferably, it refers to mammals comprising human. The subject may be a patient who needs treatment of diseases.

Meanwhile, homoisoflavanone and a salt thereof of the present invention have effect on preventing or treating allergic diseases. Accordingly, the present invention provides a pharmaceutical composition for preventing and treating allergic diseases comprising homoisoflavanone or a pharmaceutically acceptable salt thereof. In addition, the present invention provides use of homoisoflavanone or a pharmaceutically acceptable salt thereof for the preparation of reagent for preventing and treating allergic diseases. In addition, the present invention provides a method for treating allergic diseases comprising administering to a subject in need thereof an effective amount of homoisoflavanone or a pharmaceutically acceptable salt thereof.

As used herein, the term ‘allergic disease’ refers to a hypersensitivity of the human body to an external substance, i.e. a disorder occurring due to a hypersensitive response of the immune system to that substance. Non-limiting examples of the allergic disease may include allergic dermatitis, atopic dermatitis, allergic rhinitis, allergic asthma, anaphylactic shock, allergic conjunctivitis, or the like. The allergic dermatitis may include atopic dermatitis, allergic contact dermatitis, allergic urticaria, drug eruption, and so forth.

Regarding the effective ingredients, formulations, and effective amount of the pharmaceutical composition of the present invention for preventing and treating the allergic diseases, the skilled person in the art may understand them by modifying, changing or supplement the above-mentioned description.

Meanwhile, the present invention provides a cosmetic composition for preventing or improving skin inflammation comprising homoisoflavanone or a salt thereof of the present invention. In addition, the present invention provides use of homoisoflavanone or a salt thereof for the preparation of reagent for preventing or improving skin inflammation. In addition, the present invention provides a method for improving skin inflammation comprising administering to a subject in need thereof an effective amount of homoisoflavanone or a salt thereof.

The symptoms of skin inflammation may include common skin inflammation, acute/chronic eczema, contact dermatitis, atopic dermatitis, seborrheic dermatitis, lichen simplex chronicus, intertrigo, exfoliative dermatitis, papular urticaria, lichen planus, acute/chronic lichenoid dermatitis, irritant dermatitis, parapsoriasis, pityriasis rosea, panniculitis, solar dermatitis, exfoliative dermatitis, mastocytosis, psoriasis or acne. The cosmetic composition according to the present disclosure may be prepared into any formulations for topical application according to the methods known in the art using homoisoflavanone or a salt thereof as an active ingredient and further using a cosmetically and/or dermatologically acceptable excipient. For example, the cosmetic composition according to the present disclosure may be prepared into solution, gel, solid, emulsion, suspension, microemulsion, microcapsule, microgranule, ionic and/or non-ionic vesicular dispersion, cream, lotion, powder, ointment, spray or stick, although not limited thereto.

The cosmetically and/or dermatologically excipient may include a skin emollient, an emulsifier, a thickener or a solvent.

The skin emollient is used to soften, soothe, coat, smoothen or moisturize the skin. The skin emollient may be any one known in the art. For example, it may be petroleum-based, fatty acid ester type, alkyl ethoxylate type, fatty acid ester ethoxylate type, fatty alcohol type, polysiloxane type or a mixture thereof. Further, common skin emollients such as propylene glycol, butylene glycol, glycerine, triethylene glycol, spermaceti, wax, fatty acid, fatty alcohol ether, glyceride, acetoglyceride, ethoxylated glyceride, polyhydroxy alcohol, lanolin and derivatives may be included therein. The emulsifier serves to mix the water phase and the oil phase of the cosmetic composition. The emulsifier may be one or more of a non-ionic, anionic or cationic type. The emulsifier may be determined whether the emulsion is a water-in-oil emulsion or an oil-in-water emulsion. Non-limiting suitable examples of the emulsifier include sorbitan trioleate, sorbitan tristearate, glycerol monooleate, glycerol monostearate, glycerol monolaurate, sorbitan sesquioleate, sorbitan monooleate, sorbitan monostearate, ethylene, beeswax derivatives of polyoxyethylene sorbitol, PEG 200 dilaurate, PEG 200 monostearate, PEG 400 dioleate, sorbitan monopalmitate, polyoxyethylene monostearate, polyoxyethylene sorbitan monostearate, or the like.

The thickener may include crosslinked carboxypolymethylene polymer, ethyl cellulose, polyethylene glycol, gum tragacanth, gum karaya, xanthan gum, bentonite, hydroxyethyl cellulose and hydroxypropyl cellulose.

The solvent may be purified water, alcohol or a mixture of purified water with alcohol.

Non-limiting examples of further cosmetic additives that may be optionally used include a preservative such as p-hydroxybenzoate ester, an antioxidant such as butylhydroxytoluene, ascorbic acid and its derivatives, and tocopherol and its derivatives, a wetting agent such as glycerol, sorbitol, 2-pyrrolidone-5-carboxylate, dibutyl phthalate, gelatin and polyethylene glycol, a pH buffer such as acetate, phosphate, citrate, triethanolamine and carbonate, a wax such as beeswax and paraffin, a thickener, an activity enhancer, a colorant, a fragrance, or the like.

In the cosmetic composition of the present invention, the preferable amount of homoisoflavanone of the present invention is about 0.001 to 10 weight %.

Meanwhile, homoisoflavanone and a salt thereof of the present invention have effect on preventing or improving allergy. Accordingly, the present invention provides a cosmetic composition for preventing and improving allergy comprising homoisoflavanone and a salt thereof. In addition, the present invention provides use of homoisoflavanone or a salt thereof for the preparation of reagent for preventing and improving allergy. In addition, the present invention provides a method for improving allergy comprising administering to a subject in need thereof an effective amount of homoisoflavanone or a salt thereof.

As used herein, the ‘allergy’ refers to hypersensitivity of the body to a certain substance, i.e. a hypersensitive reaction of the human immune system to an external substance. For example, the allergy may be allergic dermatitis. The allergic dermatitis may include atopic dermatitis, allergic contact dermatitis, allergic urticaria, drug eruption, etc.

### Advantageous Effects

Accordingly, the present disclosure provides a new use of homoisoflavanone or a salt thereof for the prevention and treatment of inflammatory diseases, allergic diseases, skin inflammations or allergies. Since the homoisoflavanone of the present or a salt thereof inhibits production of reactive oxygen species, COX-2 and pro-inflammatory cytokines involved in inflammatory reactions and degranulation of mast cells, and also suppresses ear edema and hyperplasia in animal experiments, it is effective in suppressing inflammatory diseases. And since the homoisoflavanone or a salt thereof inhibits production of leukotriene B4 and C4 and histamine and proliferation of T lymphocytes, it is effective in allergic diseases. Therefore, it can be used to prevent, treat and improve these diseases.

### DESCRIPTION OF DRAWINGS

FIG. 1 shows inhibition effect of homoisoflavanone represented by Chemical Formula 1 on intracellular reactive oxygen species (Con: non-treated group; HIF: test group A; UVB 20 mJ/cm²: control group; U+H: test group B);

FIG. 2 shows inhibition effect of homoisoflavanone on COX-2 expression (a) in human keratinocytes and on signal transduction of PLA2 (b) MAPK (c and d) and NF-κB (e) (pcPLA2: phosphorylated PLA2, pERK: phosphorylated ERK, pp38: phosphorylated P38);

FIG. 3 shows inhibition effect of homoisoflavanone on the pro-inflammatory cytokines IL-6, IL-8 and TNF-α;

FIG. 4 shows inhibition effect of homoisoflavanone and dexamethasone against skin damage induced by phorbol 12-myristate 13-acetate;

FIG. 5 shows inhibition effect of homoisoflavanone and dexamethasone against skin damage induced by arachidonic acid;

FIG. 6 shows inhibition effect of homoisoflavanone on release of leukotriene in human mast cells and on the pro-inflammatory cytokines IL-6 and IL-8;

FIG. 7 shows inhibition effect of homoisoflavanone on COX-2 expression (a), 5-LOX translocation (b) and cPLA₂ phosphorylation (c) and maintenance of ERK phosphorylation (c) in human mast cells;

FIG. 8 shows inhibition effect of homoisoflavanone on degranulation in mast cells (a: transmission electron microscopic images, b: release of histamine, c: release of hexosaminidase);

FIG. 9 shows inhibition effect of homoisoflavanone on T cell proliferation of a mouse; and

FIG. 10 shows effect of homoisoflavanone on irritant dermatitis (a: UVB, b: croton oil).

### MODE FOR INVENTION

The examples and experiments will now be described.

The following examples and experiments are for illustrative purposes only and not intended to limit the scope of this disclosure.

### Example 1

### Inhibition Effect Against Intracellular Reactive Oxygen Species (ROS) Induced by UV Radiation

<1-1> Inhibition Effect of Homoisoflavanone Against Intracellular Reactive Oxygen Species

Human keratinocyte cell line HaCaT cells (acquired from Professor N. Fusenig of the German Cancer Research Center) were inoculated on a 60-mm culture dish (5×10⁵ cells; 1×10⁶ cells for a 100-mm culture dish) and cultured for 24 hours in Dulbecco's modified Eagle's medium (DMEM) containing 10% fetal bovine serum (FBS).

Then, after treating with homoisoflavanone of Chemical Formula 1 (R=OCH₃) at a concentration of 1 μg/mL for 1 hour, the cells were washed 3 times with phosphate buffered saline (PBS). In order to generate reactive oxygen species (ROS), the cells were irradiated with UV at 100 J/m² using a UV lamp (FSX 24 T12/UVB/HO, Pansol™, USA). Immediately after the UV radiation, the cells were washed again with PBS and further cultured for 4 hours in DMEM containing 1% FBS, while treating with homoisoflavanone at the same concentration. Then, after treating for 30 minutes in Hank's balanced salt solution (HBSS, BioWhittaker, Cambrex, USA) with 25 μM 2,7-dichlorofluorescein diacetate (DCFH-DA), the cells were washed 3 times with PBS.

Thereafter, the ROS induced by UV were measured using a fluorescence microscope (Carl Zeiss, USA). Test groups were subdivided as follows: non-treated group—treated with neither UV nor homoisoflavanone; test group A—treated with homoisoflavanone only; control group—treated with UV only; test group B—treated with UV and homoisoflavanone.

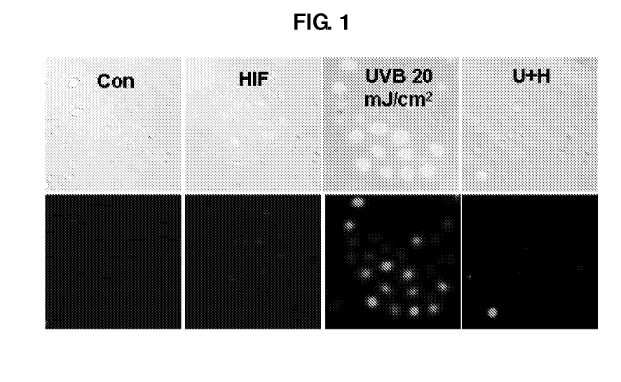

As seen in FIG. 1, when homoisoflavanone was treated before the UV radiation (test group B, U+H), generation of intracellular ROS decreased significantly as compared to when only UV was irradiated (control group, UVB 20 mJ/cm²). Thus, it was confirmed that homoisoflavanone is effective in inhibiting the generation of ROS induced by UV. This suggests that the homoisoflavanone compound of the present invention has an antioxidative effect.

### Example 2

### Inhibition Effect Against COX-2 Expression Induced by UV

<2-1> Inhibition Effect of Homoisoflavanone Against COX-2 Expression Induced by UV

In order to investigate the effect of homoisoflavanone on COX-2 expression induced by UV in human keratinocytes, HaCaT cells (5×10⁵ cells) were inoculated on a 60-mm culture dish and cultured for 24 hours in DMEM containing 10% FBS.

Then, the cells were starved by culturing for 24 hours in DMEM containing 1% FBS. Then, after treating with homoisoflavanone at 1 μg/mL for 1 hour, the cells were washed 3 times with PBS and irradiated with UV at 100 J/m² to induce COX-2 expression. Immediately after the UV radiation, the cells were washed again with PBS and further cultured for 16 hours in DMEM containing 1% FBS, while treating with homoisoflavanone at the same concentration.

Thereafter, Western blotting was performed as follows in order to measure the degree of COX-2 expression in the cultured cells. The cells of each group were lysed using RIPA buffer containing 2 mM EDTA, 137 mM NaCl, 20 mM Tris-HCl (pH, 8.0), 1 mM sodium vanadate, 10 mM NaF, 1 mM phenylmethanesulfonyl fluoride (PMSF), 1% Triton X-100, 10% glycerol and a protease inhibitor cocktail, and subjected to SDS-PAGE electrophoresis on 10% acrylamide gel to separate proteins, which were then transferred to a nylon membrane and separated using goat anti-COX-2 antibody (SantaCruz, USA) and anti-goat IgG-horse radish peroxidase (HRP) conjugation antibody (1:10000, Zymed) as secondary antibody. After detection using an ECL (Amersham, USA), the result was imaged using RAS 3000 Imaging system (Fujifilm, Japan).

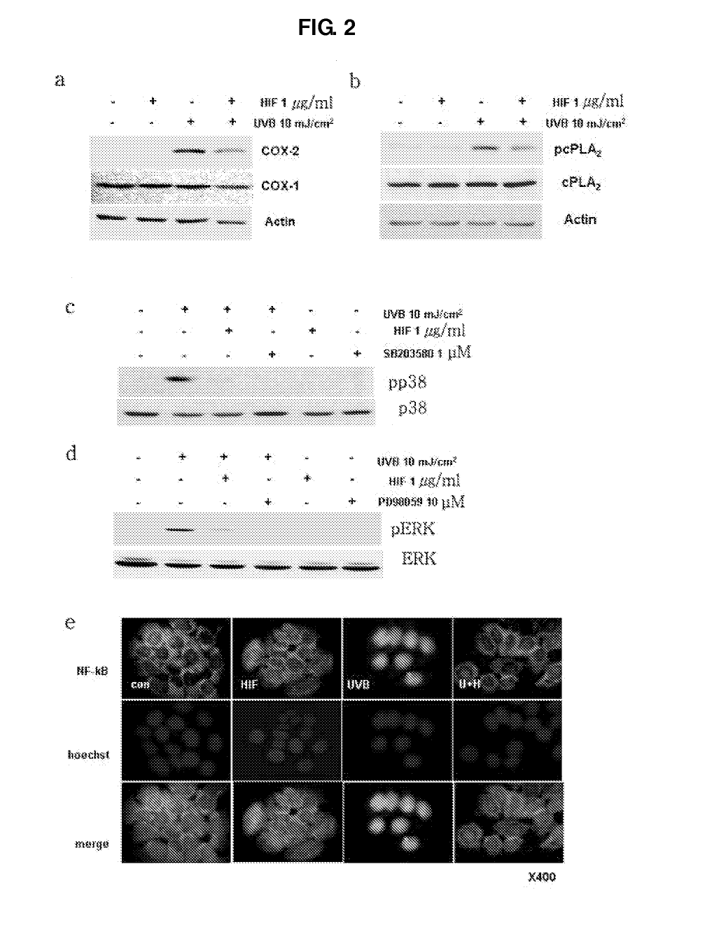

As seen in FIG. 2 a, the control group (treated only with UV) showed increased COX-2 expression due to the UV radiation, which was decreased by homoisoflavanone treatment.

<2-2> Confirmation of the Mechanism of the Inhibition COX-2 Expression

In order to study how the COX-2 expression induced by UV radiation in human keratinocytes is inhibited by homoisoflavanone, signaling of MAPK and the transcription factor NF-κB was investigated.

MAPKs are mainly involved in intracellular inflammation, apoptosis, carcinogenesis, or the like and are related to cell damage induced by UV radiation (Peus D, Vasa R A, Beyerle A, Meves A, Krautmacher C, Pittelkow M R. UVB activates ERK1/2 and p38 signaling pathways via reactive oxygen species in cultured keratinocytes. J. Invest. Dermatol. 1999; 112: 7516.). Also, the transcription factor NF-κB is involved in intracellular inflammatory reactions and regulates various intracellular factors including COX-2 (Paik J, Lee J Y, Hwang D. Signaling pathways for TNFα-induced COX-2 expression: mediation through MAP kinases and NF-κB, and inhibition by certain nonsteroidal anti-inflammatory drugs. Adv. Exp. Med. Biol. 2002; 507: 503-508).

In order to identify the intracellular factors affecting COX-2 expression that are affected by homoisoflavanone, Western blotting was carried out in the same manner as in Example 2-1 except for treating with homoisoflavanone at 1 μg/mL, irradiating UV for 45 minutes and using rabbit anti-p-cytosolic phospholipase A2 (cPLA₂) antibody, rabbit anti-cPLA₂ antibody, rabbit anti-ERK antibody, rabbit anti-p-ERK antibody, rabbit anti-p38 antibody or rabbit anti-p-p38 antibody and using anti-rabbit IgG-HRP conjugation antibody (Zymed) as secondary antibody. Positive control groups were treated with 1 μM SB203580 (A.G. Scientific, Inc.) as p38 inhibitor or 10 μM PD98059 (A.G. Scientific, Inc.) as MEK 1 inhibitor instead of homoisoflavanone.

As seen in FIGS. 2 b (cPLA₂), 2c (p38) and 2d (ERK), phosphorylation of cPLA₂, p38 and ERK was inhibited by homoisoflavanone.

<2-3> Confirmation of the Mechanism of the Inhibition COX-2 Expression

In order to investigate whether the transcription factor NF-κB is activated, human keratinocytes HaCaT cells were immunocytochemically stained as follows.

After inoculating the cells on a 14-mm culture slide, the cells were cultured in DMEM containing 10% FBS for 24 hour. Then, the cells were starved by culturing for 24 hours in DMEM containing 1% FBS. Then, after treating with homoisoflavanone at 1 μg/mL for 1 hour, the cells were washed 3 times with PBS and irradiated with UV at 100 J/m². Immediately after the UV radiation, the cells were washed again with PBS and further cultured for 4 hours in DMEM containing 1% FBS while treating with homoisoflavanone at the same concentration. Then, the cells were fixed with methanol for 5 minutes and then subjected to immunocytochemical staining.

The fixed cells were blocked with 10% normal goat serum and then washed 3 times with PBS. Then, after fluorescence staining NF-κB using rabbit anti-NF-κB antibody (SantaCruz, USA) and Alexa 488-conjugated anti-rabbit IgG secondary antibody (Invitrogen, USA), counter staining was carried out using Hoechst (Sigma, St. Louis, Mo.).

As seen in FIG. 2e, NF-κB was activated by UV radiation and translocated into the nucleus. In contrast, in the test group B, which had been treated with homoisoflavanone prior to the UV radiation, NF-κB existed in the cytoplasm as in the non-treated group or the test group A which had been treated only with homoisoflavanone. Thus, it can be seen that homoisoflavanone inhibits the migration of NF-κB into the nucleus induced by UV radiation.

### Example 3

### Inhibition Effect of Homoisoflavanone Against Increase of Pro-Inflammatory Cytokines Induced by UV Radiation

UV radiation leads to increase of cytokines such as interleukin-6 (IL-6), IL-8 and tumor necrosis factor-α (TNF-α) in skin tissue, thereby mediating inflammatory reactions. In order to investigate the effect of homoisoflavanone thereon, experiment was carried out as follows using human keratinocytes.

1×10⁶ HaCaT cells were inoculated on a 100-mm culture dish and cultured in DMEM containing 10% FBS for 24 hour. Then, the cells were starved by culturing for 24 hours in DMEM containing 1% FBS. Then, after treating with homoisoflavanone at 1 μg/mL for 1 hour, the cells were washed 3 times with PBS and irradiated with UV at 100 J/m² to induce increase of cytokines. Immediately after the UV radiation, the cells were washed again with PBS and further cultured for 2 or 5 hours in DMEM containing 1% FBS which had been treated with homoisoflavanone at the same concentration.

Thereafter, the degree of mRNA expression of IL-6, IL-8 and TNF-α, which is mainly induced in skin by UV radiation, was measured by RT-PCR in order to investigate the inhibitory effect against the pro-inflammatory cytokines.

After extracting RNA from the cells of each test group using TRIzol (Invitrogen, USA), cDNA was prepared by carrying out PCR (PTC-225 Peltier thermal cycler, MJ Research, USA). Then, the mRNA expression of IL-6, IL-8 and TNF-α was measured through real-time PCR (Roter Gene 6000 series, Corbett Research, Australia) using the cDNA as template and using commercially available IL-6, IL-8 and TNF-α primers. The expression level of the pro-inflammatory cytokines was compared relative to the non-treated group.

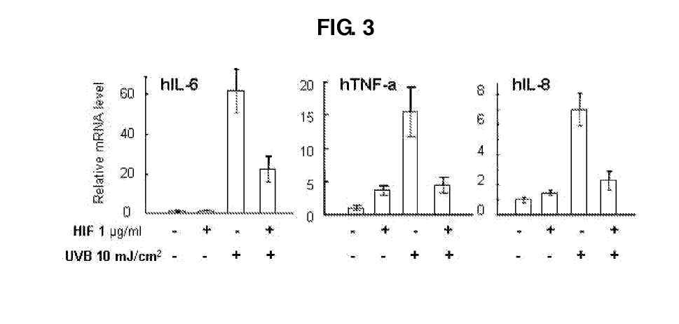

As seen in FIG. 3, UV ration to human keratinocytes (control group) resulted in increased expression of the pro-inflammatory cytokines IL-6(human IL-6, hIL-6), IL-8 (human IL-8, hIL-8) and TNF-α (human TNF-α, hTNF-α). In contrast, the test group B which had been treated with homoisoflavanone showed decreased expression of IL-6, IL-8 and TNF-α as compared to the control group. Thus, it can be seen that homoisoflavanone remarkably reduces the expression of -6, IL-8 and TNF-α induced by UV radiation.

### Example 4

### Effect of Homoisoflavanone on Ear Edema Induced by Phorbol 12-myristate 13-acetate in Animal Model

In order to confirm the in vitro test result on the human keratinocytes HaCaT cells in an animal model, experiment was performed as follows using hairless mouse (SKH-1). Test groups are as follows: non-treated group—treated not with phorbol 12-myristate 13-acetate but only with acetone (TPA); test group A—treated with 1 μg/10 μL homoisoflavanone or 50 μg/10 μL dexamethasone; control group (TPA)-treated with 100 μM phorbol 12-myristate 13-acetate; test group B—treated with 1 μg/10 μL homoisoflavanone or 50 μg/10 μL dexamethasone as well as 100 μM phorbol 12-myristate 13-acetate.

One hour before treating with phorbol 12-myristate 13-acetate, 1 μg/10 μL homoisoflavanone or 50 μg/10 μL dexamethasone was applied on the front and rear parts of the ear, 5 μL each. Then, 6 hour after the application of phorbol 12-myristate 13-acetate, ear thickness and weight were measured.

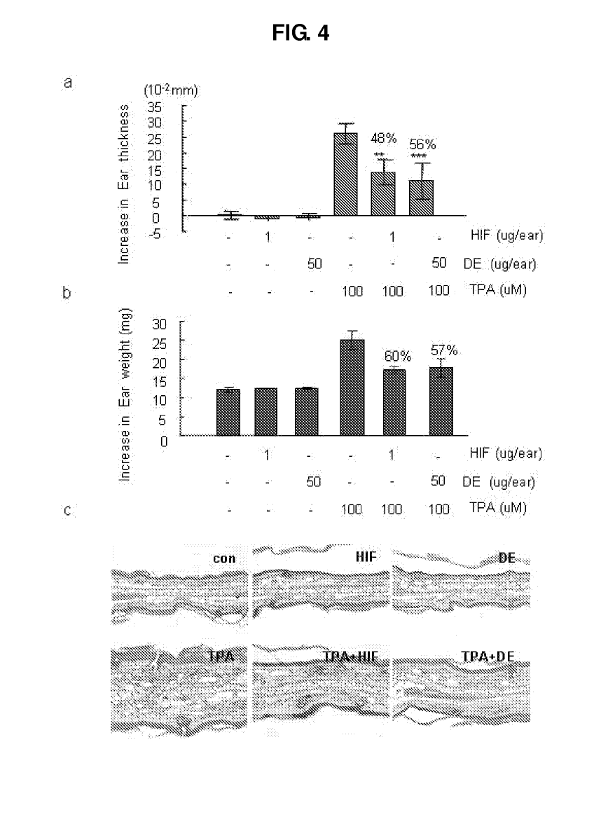

As seen in FIGS. 4a and 4b, the control group (TPA) showed increase in ear thickness and weight. In contrast, the mouse to which homoisoflavanone or dexamethasone had been applied and then phorbol 12-myristate 13-acetate was applied (test group B, TPA+HIF or TPA+DE) showed decrease in ear thickness and weight.

In order to investigate overall change in the skin tissue, hematoxylin-eosin staining was carried out. As seen in FIG. 4c, the non-treated group (TPA) showed hyperplasia in the epidermis and increased infiltration of inflammatory cells. In contrast, the mouse to which homoisoflavanone or dexamethasone had been applied and then phorbol 12-myristate 13-acetate was applied (test group B, TPA+HIF or TPA+DE) showed significantly reduced hyperplasia in the epidermis.

This result suggests that, like the currently used dexamethasone, homoisoflavanone is effective in treating skin inflammations caused by phorbol 12-myristate 13-acetate.

### Example 5

### Effect of Homoisoflavanone on Ear Edema Induced by Arachidonic Acid in Animal Model

Experiment was carried out as follows using BALB/c mouse. Test groups are as follows: non-treated group—treated not with arachidonic acid (AA) but only with acetone; control group (AA)-treated with arachidonic acid; test group A—treated with 1 μg/10 μL homoisoflavanone or 50 μg/10 μL dexamethasone as well as 100 mg/mL 10 μL arachidonic acid.

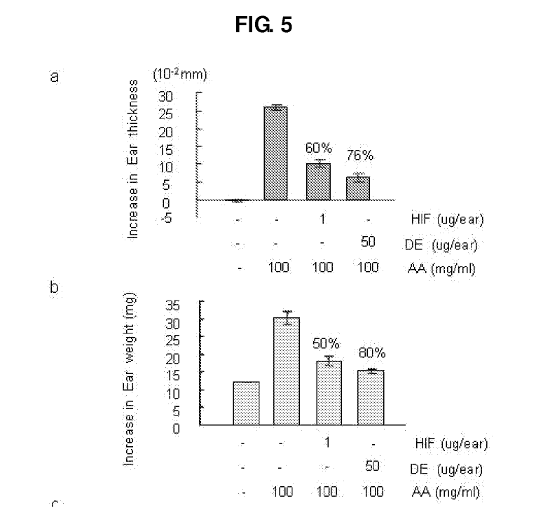

One hour before treating with arachidonic acid, 1 μg/10 μL homoisoflavanone or 50 μg/10 μL dexamethasone was applied on the front and rear parts of the ear, 5 μL each. Then, 1 hour after applying 100 mg/mL 10 μL arachidonic acid, ear thickness and weight were measured. As seen in FIGS. 5a and 5b, the control group (AA) showed increase in ear thickness and weight. In contrast, the mouse to which homoisoflavanone or dexamethasone had been applied and then arachidonic acid was applied (test group A, AA+HIF or AA+DE) showed decrease in ear thickness and weight.

This result suggests that, like the currently used dexamethasone, homoisoflavanone is effective in treating skin inflammations caused by arachidonic acid.

### Example 6

### Effect of Homoisoflavanone on Inflammatory Factors Induced by Phorbol 12-myristate 13-acetate and A23187 in Mast Cells

<6-1> Inhibition Effect of Homoisoflavanone Against Release of Leukotriene Induced by Phorbol 12-myristate 13-acetate and A23187

Phorbol 12-myristate 13-acetate and A23187 lead to degranulation of human mast cells (HMC), causing the mast cells to release not only their histamine or serotonin but also newly produced inflammatory factors such as pro-inflammatory cytokines, prostaglandins and leukotrienes. It was investigated whether homoisoflavanone has anti-inflammatory effect in mast cells as in Examples 4 and 5. In order to investigate the effect of homoisoflavanone on the release of leukotriene induced by phorbol 12-myristate 13-acetate and A23187 in HMC-1, 1×10⁶ HMC-1 cells were inoculated on a 100-mm culture dish and cultured in Iscove's modified Dulbecco's medium (IMDM, GIBCO) containing 10% FBS for 48 hours. Then, after treating with homoisoflavanone at 2 μg/mL or with mizolastine at 10 μg/mL for comparison for 1 hour, the cells were treated with 50 nM phorbol 12-myristate 13-acetate and 1 μM A23187 to induce release of leukotriene, and then further cultured for 21 hours. Then, the supernatant was collected and the quantity of released leukotriene B4 and C4 was measured using a leukotriene B4, C4 enzyme immunoassay kit (Sapphire Bioscience, Australia).

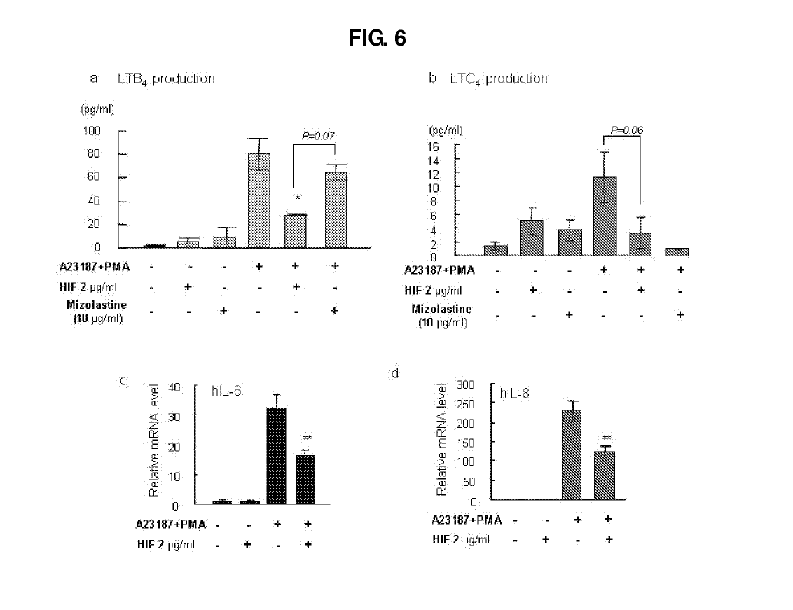

As seen in FIG. 6a, the release of leukotriene B4 induced by phorbol 12-myristate 13-acetate and A23187 was inhibited by homoisoflavanone by 70%, whereas it was inhibited by 20% by mizolastine. When homoisoflavanone or mizolastine was treated alone, there was no appreciable difference from the non-treated group. As seen in FIG. 6b, the release of leukotriene C4 was inhibited by 90% or more by both homoisoflavanone and mizolastine.

<6-2> Inhibition Effect of Homoisoflavanone Against Release of Pro-Inflammatory Cytokines Induced by Phorbol 12-myristate 13-acetate and A23187

In order to investigate the effect of homoisoflavanone on the release of pro-inflammatory cytokines induced by phorbol 12-myristate 13-acetate and A23187 in HMC-1, 5×10⁵ HMC-1 cells were inoculated on a 60-mm culture dish and cultured in IMDM containing 10% FBS for 48 hours. Then, after treating with homoisoflavanone at 2 μg/mL, the cells were treated with 50 nM phorbol 12-myristate 13-acetate and 1 μM A23187 to induce release of pro-inflammatory cytokines, and then further cultured for 5 hours.

Then, the degree of mRNA expression of IL-6 and IL-8, which is mainly induced in skin by UV radiation, was measured by RT-PCR in order to investigate the inhibitory effect against the pro-inflammatory cytokines.

After extracting RNA from the cells of each test group using TRIzol, cDNA was prepared by carrying out PCR using the RNA as a template. Then, the mRNA expression of IL-6 and IL-8 was measured through real-time PCR using the cDNA as template and using commercially available IL-6 and IL-8 primers. The expression level of the pro-inflammatory cytokines was compared relative to the non-treated group.

As seen in FIGS. 6c and d, the release of IL-6 and IL-8 induced by phorbol 12-myristate 13-acetate and A23187 was inhibited by homoisoflavanone by 50%.

Thus, homoisoflavanone, having inhibitory effect against release of the pro-inflammatory factor leukotriene B4 and mRNA expression of IL-6 and IL-8 as well as against release of leukotriene C4, is expected to have anti-allergic effect in addition to the anti-inflammatory effect.

### Example 7

### Effect of Homoisoflavanone on COX-2 Expression Induced by Phorbol 12-myristate 13-acetate and A23187 in Mast Cells

<7-1> Inhibition Effect of Homoisoflavanone Against COX-2 Expression Induced by phorbol 12-myristate 13-acetate and A23187

In order to investigate the effect of homoisoflavanone on COX-2 expression induced by phorbol 12-myristate 13-acetate and A23187 in human mast cells, 1×10⁶ HMC-1 cells were inoculated on a 100-mm culture dish and cultured in IMDM containing 10% FBS for 48 hours. Then, after treating with homoisoflavanone at 2 μg/mL or with celecoxib at 60 μM for comparison for 1 hour, the cells were treated with 50 nM phorbol 12-myristate 13-acetate and 1 μM A23187 to induce COX-2 expression, and then further cultured for 21 hours. Then, Western blotting was carried out in the same manner as in Example 2-1 except for using goat anti-COX-2 antibody (SantaCruz) and using anti-rabbit IgG-HRP conjugation antibody (Zymed) as secondary antibody.

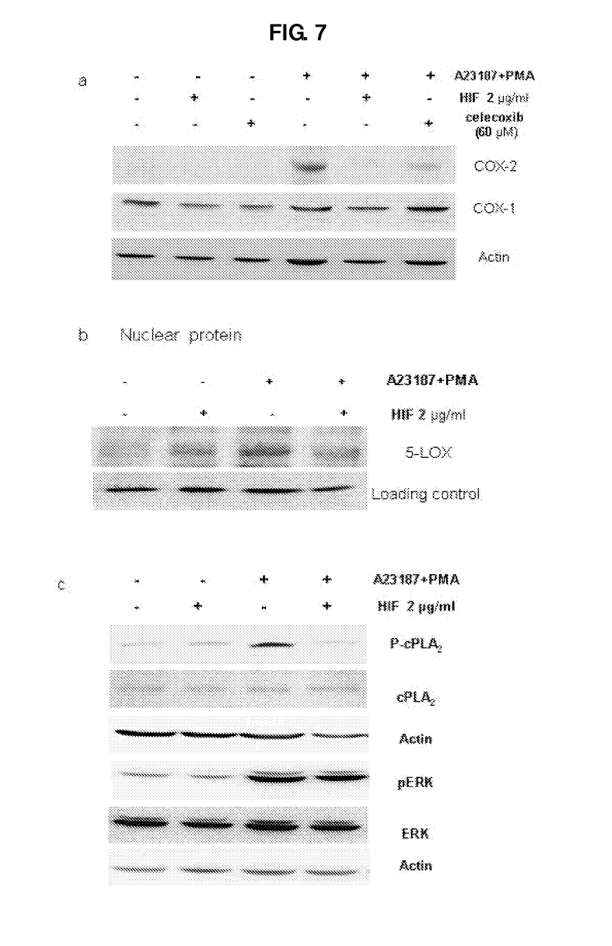

As seen in FIG. 7a, the COX-2 expression induced by phorbol 12-myristate 13-acetate and A23187 was inhibited by 80% by homoisoflavanone and by 50% by celecoxib. Thus, homoisoflavanone will be more effective than the currently marketed celecoxib.

<7-2> Inhibition Effect of Homoisoflavanone Against 5-LOX Translocation to Nuclear Membrane Induced by phorbol 12-myristate 13-acetate and A23187

In order to investigate the effect of homoisoflavanone on the migration of 5-LOX from the cytoplasm toward the nuclear membrane induced by phorbol 12-myristate 13-acetate and A23187 in human mast cells, 1×10⁶ HMC-1 cells were inoculated on a 100-mm culture dish and cultured in IMDM containing 10% FBS for 48 hours. Then, after treating with homoisoflavanone at 2 μg/mL, the cells were treated with 50 nM phorbol 12-myristate 13-acetate and 1 μM A23187 to induce migration of 5-LOX to the nuclear membrane, and then further cultured for 5 hours.

Then, in order to measure the migration of 5-LOX to the nuclear membrane, nuclear membrane proteins were isolated first. The cells of each test group were lysed with a buffer containing 10 mM HEPES/KOH, 0.1 mM EDTA, 10% NP-40, 10 mM KCl, 1 mM DTT, 0.5 mM PMSF and a protease inhibitor cocktail on ice for 10 minutes. After centrifugation, the supernatant (cytoplasmic proteins) was separated from the pellets (nuclear membrane proteins). The cytoplasmic proteins were kept at −80° C. and the pellets were lysed with a buffer containing 10 mM HEPES/KOH, 0.1 mM EDTA, 10% NP-40, 300 mM NaCl, 1 mM DTT, 0.5 mM PMSF and a protease inhibitor cocktail by shaking on ice for 30 minutes. Then, after centrifugation, the supernatant (nuclear proteins) was separated. The obtained cytoplasmic proteins and nuclear proteins were subjected to SDS-PAGE electrophoresis on 8% acrylamide gel, which were then transferred to a nylon membrane and subjected to Western blotting in the same manner as in Example 2-1 except for using rabbit anti-5-LOX antibody (Cayman) and using anti-rabbit IgG-HRP conjugation antibody (Zymed) as secondary antibody.

As seen in FIG. 7b, the migration of 5-LOX toward the nuclear membrane induced by phorbol 12-myristate 13-acetate and A23187 was inhibited by homoisoflavanone.

<7-3> Confirmation of Mechanism by which Homoisoflavanone Inhibits COX-2 Expression and 5-LOX Translocation Induced by Phorbol 12-Myristate 13-Acetate and A23187

In human mast cells, phorbol 12-myristate 13-acetate and A23187 result in increased calcium concentration, which leads to migration of cPLA₂ from the cytoplasm into the nucleus, resulting in release of arachidonic acid, which induces release of prostaglandin or leukotriene by such enzymes as COX or LOX, thereby inducing inflammatory or allergic reactions.

It is reported that the phosphorylation of cPLA₂ in mast cells is mediated by ERK. Thus, the effect of homoisoflavanone on phosphorylation of cPLA₂ and ERK was investigated.

1×10⁶ HMC-1 cells were inoculated on a 100-mm culture dish and cultured in IMDM containing 10% FBS for 48 hours. Then, after treating with homoisoflavanone at 2 μg/mL or with celecoxib at 60 μM for comparison for 1 hour, the cells were treated with 50 nM phorbol 12-myristate 13-acetate and 1 μM A23187, and then further cultured for 4 hours for phosphorylation of cPLA₂ and for 30 minutes for phosphorylation of ERK. Then, Western blotting was carried out in the same manner as in Example 2-1 except for using rabbit anti-p-cytosolic phospholipase A2 (cPLA₂) antibody, rabbit anti-cPLA₂ antibody, rabbit anti-ERK antibody and rabbit anti-p-ERK antibody (Cell Signaling) and using anti-rabbit IgG-HRP conjugation antibody (Zymed) as secondary antibody.

As seen in FIG. 7c, cPLA₂ phosphorylation was induced by phorbol 12-myristate 13-acetate and A23187. The group that had been treated with homoisoflavanone inhibited cPLA₂ phosphorylation like the non-treated group. But, it had no effect on ERK phosphorylation. Thus, it was confirmed that homoisoflavanone inhibits COX-2 expression and 5-LOX translocation to the nuclear membrane by inhibiting the phosphorylation of cPLA₂, not that of ERK.

### Example 8

### Effect of Homoisoflavanone on Degranulation Induced by Phorbol 12-myristate 13-acetate and A23187 in Mast Cells

Phorbol 12-myristate 13-acetate and A23187 induce degranulation in human mast cells, resulting in release of such substances as histamine and serotonin and inducing allergic reactions. Since the release of pro-inflammatory cytokines is related with degranulation, the effect of homoisoflavanone on degranulation was investigated.

In order to investigate the effect of homoisoflavanone on COX-2 expression induced by phorbol 12-myristate 13-acetate and A23187 in HMC-1, 1×10⁵ HMC-1 cells were inoculated on a 6-well plate and cultured in IMDM containing 10% FBS for 48 hours. Then, after treating with homoisoflavanone at 2 μg/mL for 1 hour, the cells were treated with 50 nM phorbol 12-myristate 13-acetate and 1 μM A23187 to induce COX-2 expression, and then further cultured for 6 hours. Then, the cells were fixed with 2.5% glutaraldehyde and frozen with liquid nitrogen. The cells were detached from the plate by applying heat, sliced into 60-80 μm, attached on grids, and observed with a transmission electron microscope (TEM).

In order to measure histamine released during the degranulation, the cell supernatant was collected and subjected to measurement using a histamine enzyme assay kit (Cayman, USA) according to the manufacturer's instructions. First, the collected supernatant was anti-histamine derivatized and reacted with histamine-AChE tracer. Then, after washing at least 5 times followed by visualization for 60-90 minutes, absorbance was measured at 414 nm.

Further, the effect of homoisoflavanone on degranulation of rat mast cells induced by antigen was investigated. 5×10⁴ rat mast cells were inoculated on a 6-well plate and cultured in MEM containing 15% FBS for 24 hours. Then, after reacting with 1 μg/mL DNP-IgE for 16 hours and then removing DNP-IgE by washing with PBS, the cells were cultured in 15% MEM for 6 hours. After pretreating with homoisoflavanone in Tyrode's solution for 30 minutes, the cells were treated with 10 ng/mL DNP-human serum albumin (HSA) to induce degranulation by transmitting the signals induced by the antigen. 30 minutes later, the supernatant was collected and the pellets were lysed with 0.5% triton X-100 (500 μL). The supernatant and the lysed pellets (70 μL) were incubated at 37° C. for 90 minutes in 0.1 M sodium citrate solution (pH 4.5) containing 8 mM p-nitrophenyl-N-acetyl-β-d-glucosaminide (70 μL). Then, after adding 0.2 M glycine (pH 10.7, 70 μL) to stop the reaction, absorbance was measured at 405 nm. Total enzymatic activity was calculated by the following equation.

Release amount=(Absorbance of supernatant)÷(Absorbance of supernatant+Absorbance of pellets)×100%

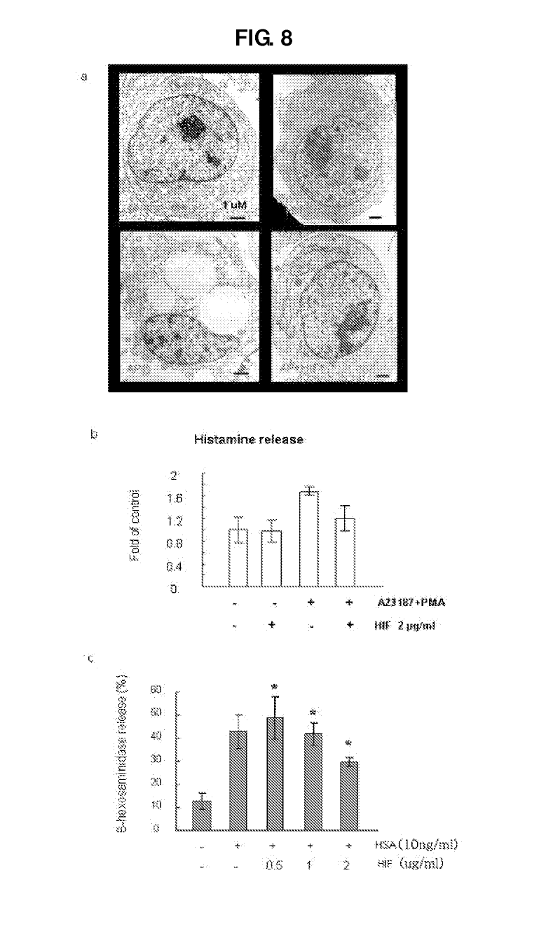

As seen in FIG. 8 a, the release of intracellular substances induced by phorbol 12-myristate 13-acetate was inhibited by homoisoflavanone. And, as seen in FIG. 8 b, the release of histamine was inhibited by homoisoflavanone by 72%. Also, as seen in FIG. 8 c, the inhibition of antigen-induced degranulation in rat mast cells by homoisoflavanone was concentration-dependent, with 50% inhibition at 2 μg/mL. Accordingly, it can be seen that homoisoflavanone which inhibits degranulation of mast cells has an anti-allergic effect.

### Example 9

### Effect of Homoisoflavanone on Mixed Lymphocyte Reaction (MLR)

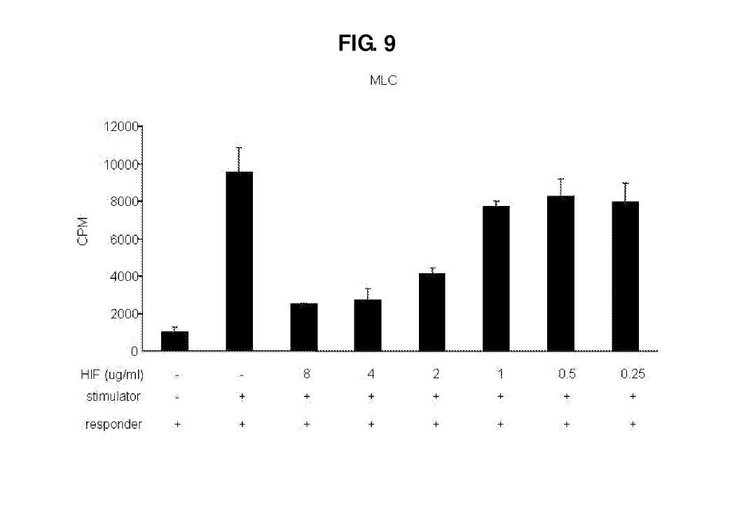

Mixed lymphocyte reaction (MLR) was carried out in order to investigate the inhibitory effect of homoisoflavanone against T cell proliferation. When mouse immune cells of different MHC classes are cultured as mixed with each other, the T cells recognize those of different MHC class as antigens and thus proliferate. One-way MLR was performed as follows. Following excision of spleens from BALB/C and C57B/C mice, the cells were individualized and red blood cells were lysed. After irradiation of 25 Gy γ-rays to BALB/C splenocytes to inactivate the BALB/C T cells, the BALB/C and C57B/C splenocytes (5×10⁵ and 2×10⁵ cells each) were mixed in a U-bottom 96-well plate. After treating with homoisoflavanone, the cells were incubated for 56 hours in an incubator of 5% CO₂ and 37° C. Then, after further adding ³[H]-thymidine (0.5) to each well, the cells were further incubated for 16 hours. Thereafter, the cells of each well were collected on filter paper and then put in a vial. After adding a scintillation solution (2 mL), counts per minute (CPM) was measured using a β-counter. As seen in FIG. 9, homoisoflavanone effectively inhibited the proliferation of T cells at concentrations 0.25 μg/mL or higher in a concentration-dependent manner.

### Example 10

### Effect of Homoisoflavanone on Ear Edema Induced by Various Stimuli (UV and Croton Oil) in Animal Model

<10-1> Effect of Homoisoflavanone on Ear Edema Induced by UV Radiation

BALE/c mice were subdivided into a non-treated group (treated with acetone only without UV radiation), a control group (irradiated with UV at 7.5 kJ/m²) and a test group (treated with 0.6 or 1 μg/10 μL homoisoflavanone and irradiated with UV at 7.5 kJ/m²). Then, the effect on ear edema induced by UV radiation was investigated.

One hour before irradiating UV at 7.5 kJ/m², 1 μg/10 μL homoisoflavanone was applied on the front and rear parts of the ear, 5 μL each. Then, 72 hours after UV radiation, ear thickness was measured.

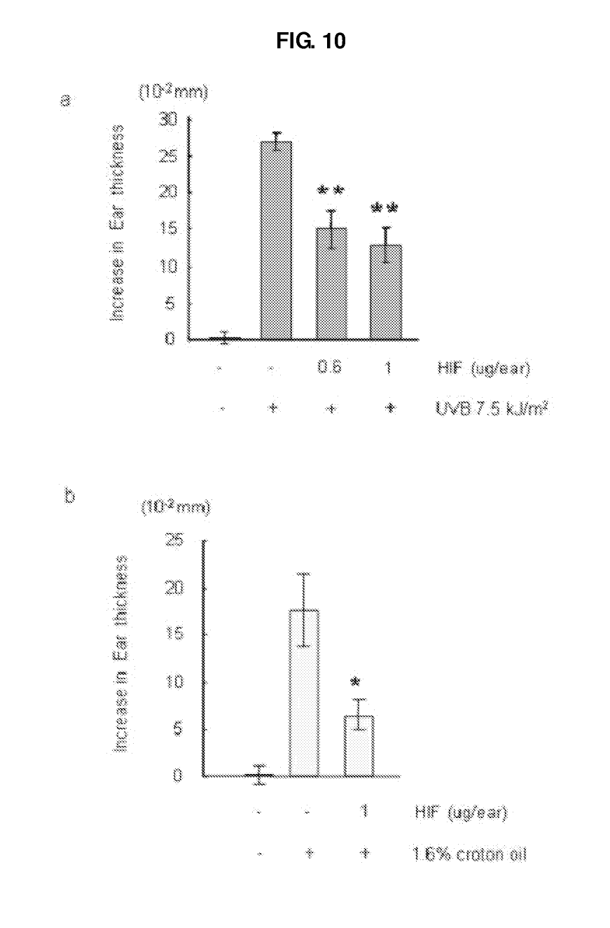

As seen in FIG. 10a, the control group (UV radiation) showed increased ear thickness. In contrast, the test group mice to which homoisoflavanone had been applied and then irradiated with UV showed concentration-dependent decrease in ear thickness of 44% (0.6 μg/ear) and 52% (1 μg/ear). Thus, it can be seen that the homoisoflavanone of the present disclosure reduces ear edema.

<10-2> Effect of Homoisoflavanone on Ear Edema Transiently Induced by Croton Oil

Hairless mice were subdivided into a non-treated group (treated with acetone only, not with croton oil), a control group (treated with 1.6% croton oil) and a test group (treated with 1 μg/10 μL homoisoflavanone and 1.6% croton oil), and the effect on ear edema induced by croton oil was investigated.

One hour before treating with 1.6% croton oil, 1 μg/10 μL homoisoflavanone was applied on the front and rear parts of the ear, 5 μL each. Then, 6 hours after treating with croton oil, ear thickness was measured.

As seen in FIG. 10b, the control group (treated with croton oil) showed increased ear thickness. In contrast, the test group to which homoisoflavanone had been applied and then treated with 1.6% croton oil (croton oil+HIF) showed decrease in ear thickness by 63%. Thus, it can be seen that homoisoflavanone can ameliorate skin edema and inflammations induced by various stimuli.

### INDUSTRIAL APPLICABILITY

As can be seen from the foregoing, the present invention provides a new use of homoisoflavanone or a salt thereof for the prevention and treatment of inflammatory diseases, allergic diseases, skin inflammations or allergies. Since the homoisoflavanone of the present or a salt thereof inhibits production of reactive oxygen species, COX-2 and pro-inflammatory cytokines involved in inflammatory reactions and degranulation of mast cells, and also suppresses ear edema and hyperplasia in animal experiments, it is effective in suppressing inflammatory diseases. And since the homoisoflavanone or a salt thereof inhibits production of leukotriene B4 and C4 and histamine and proliferation of T lymphocytes, it is effective in allergic diseases. Therefore, it can be used to prevent, treat and improve these diseases.

1. A pharmaceutical composition for preventing and treating inflammatory diseases comprising homoisoflavanone represented by Chemical Formula 1 or a pharmaceutically acceptable salt thereof as an effective ingredient. 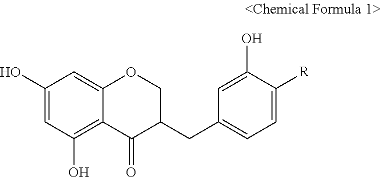
(wherein R is H, OH or OCₙH₂ₙ₊₁ (n is from 1 to 6).)

2. (canceled)

3. The composition of claim 1, wherein the inflammatory skin disease is anyone selected from the group consisting of skin inflammation, acute/chronic eczema, contact dermatitis, atopic dermatitis, seborrheic dermatitis, lichen simplex chronicus, intertrigo, exfoliative dermatitis, papular urticaria, lichen planus, acute/chronic lichenoid dermatitis, irritant dermatitis, parapsoriasis, pityriasis rosea, panniculitis, solar dermatitis, exfoliative dermatitis, mastocytosis, psoriasis or acne.

4. A pharmaceutical composition for preventing and treating allergic diseases comprising homoisoflavanone represented by Chemical Formula 1 or a pharmaceutically acceptable salt thereof as an effective ingredient.

5. The composition of claim 4, wherein the allergic diseases is anyone selected from the group consisting of allergic dermatitis, atopic dermatitis, allergic rhinitis, allergic asthma, anaphylactic shock and allergic conjunctivitis.

6. The composition of claim 5, wherein the allergic dermatitis is anyone selected from the group consisting of atopic dermatitis, allergic contact dermatitis, allergic urticaria and drug eruption.

7. A cosmetic composition for preventing and improving skin inflammation comprising homoisoflavanone represented by Chemical Formula 1 or a salt thereof as an effective ingredient.

8. The composition of claim 7, wherein the skin inflammation is anyone selected from the group consisting of skin inflammation, acute/chronic eczema, contact dermatitis, atopic dermatitis, seborrheic dermatitis, lichen simplex chronicus, intertrigo, exfoliative dermatitis, papular urticaria, lichen planus, acute/chronic lichenoid dermatitis, irritant dermatitis, parapsoriasis, pityriasis rosea, panniculitis, solar dermatitis, exfoliative dermatitis, mastocytosis, psoriasis and acne.

9. A cosmetic composition for preventing and improving allergy comprising homoisoflavanone represented by Chemical Formula 1 or a salt thereof as an effective ingredient.

10. The composition of claim 9, wherein the allergy is anyone selected from the group consisting of atopic dermatitis, allergic contact dermatitis, allergic urticaria, drug eruption.

11. Use of homoisoflavanone represented by Chemical Formula 1 or a pharmaceutically acceptable salt thereof for the preparation of an agent for preventing and treating inflammatory skin diseases.

12. A method for treating inflammatory skin diseases comprising administering to a subject in need thereof an effective amount of homoisoflavanone represented by Chemical Formula 1 or a pharmaceutically acceptable salt thereof.

13. Use of homoisoflavanone represented by Chemical Formula 1 or a pharmaceutically acceptable salt thereof for the preparation of an agent for preventing and treating allergic diseases.

14. A method for treating allergic diseases comprising administering to a subject in need thereof an effective amount of homoisoflavanone represented by Chemical Formula 1 or a pharmaceutically acceptable salt thereof.

15. Use of homoisoflavanone represented by Chemical Formula 1 or a salt thereof for the preparation of cosmetics for preventing and improving skin inflammation.

16. A method for treating skin inflammation comprising applying to a subject in need thereof an effective amount of homoisoflavanone represented by Chemical Formula 1 or a salt thereof.

17. Use of homoisoflavanone represented by Chemical Formula 1 or a salt thereof for the preparation of cosmetics for preventing and improving allergy.

18. A method for improving allergy comprising applying to a subject in need thereof an effective amount of homoisoflavanone represented by Chemical Formula 1 or a salt thereof.
